(19) United States
(12) Patent Application Publication
(10) Pub. No.: US 2026/0196303 A1
(43) Pub. Date: Jul. 9, 2026

(54) NONINVASIVE FETAL VARIANT IDENTIFICATION USING HAPLOTYPE ANALYSIS

(71) Applicant: IDENTIFAI GENETICS LTD., Rosh Haayin (IL)

(72) Inventors: Noa LISCOVITCH-BRAUER, Tel Aviv (IL); Tom RABINOWITZ, Tel Aviv (IL)

(21) Appl. No.: 19/129,659

(22) PCT Filed: Nov. 15, 2023
(86) PCT No.: PCT/IL2023/051184

§ 371 Date: May 13, 2025

(30) Foreign Application Priority Data
Nov. 15, 2022 (IL) 298246

(51) Int. Cl. G16B 30/10; C12Q 1/6881; G16B 30/20

(52) U.S. Cl. CPC G16B 30/10; C12Q 1/6881; G16B 30/20; C12Q 2600/156; C12Q 2600/172

(57) ABSTRACT

Disclosed are non-invasive methods for genotyping a fetus, comprising the analysis of sequencing data of maternal cell-free DNA (cfDNA), and parental (maternal and optionally paternal) genomic DNA (gDNA) from a pair parenting the fetus. Using a variant calling approach and optionally fragmentomics-based analysis, combined with identification of haplotypes, accurate genotype predictions are obtained.

### TECHNOLOGICAL FIELD

The present disclosure relates to the field of prenatal genetic analysis.

### REFERENCES

Byrska-Bishop et al (2022) Cell 185 (18) pp. 3426-3440
Delaneau et al (2019) Nat. Commun. 10 (1), 5436
Fan et al (2012) Nature, 487 (7407), pp 320-324
Martin et al (2016) bioRxiv, doi: 10.1101/085050
Rabinowitz et al (2019) Genome Res. 29 (3), pp. 428-438
Rabinowitz et al (2020) Comput. Struct. Biotechnol. J. 19, pp. 509-517
Scotchman et al (2020) Clin. Chem. 66 (1), pp. 53-60
Van der Auwera et al (2013) Curr. Protoc. Bioinformatics 43 (1110) 11.10.1-11.10.33
Zhang et al (2019) Nat. Med. 25 (3), p. 439

### BACKGROUND

Non-invasive prenatal testing (NIPT) is the process of assessing the health of an unborn fetus by determining the risk that the fetus will be born with deleterious genetic abnormalities. In the last decade, NIPT has emerged as a risk-free alternative to amniocentesis, enabling detection of genetic abnormalities during pregnancy. NIPT relies on the presence of cell-free fetal DNA (cffDNA) as a fraction of total cell-free DNA (cfDNA) circulating in maternal plasma from the early weeks of gestation through to birth.

In an NIPT procedure, blood is drawn from the mother, cfDNA is extracted and sequenced and is then used to gain genetic information about the fetus. Current NIPT tests are offered in clinics worldwide and can detect large, low resolution, genetic aberrations on a whole-chromosome scale, e.g., aneuploidy and large microdeletions or microduplications, or very large, specific copy number variations.

NIPT is therefore used for screening chromosomal abnormalities (e.g., trisomies, sub-chromosomal deletions and duplications), but also for monogenic disorders caused by point mutations. Commercially available NGS panels consist of up to 30 genes (Zhang et al., 2019). However, false negative results may occur in tailored tests and panels (Scotchman et al., 2020).

Genome-wide noninvasive genotyping of point mutations causing monogenic diseases requires a sequencing throughput that is often not feasible. This is especially relevant in low levels of fetal cfDNA, i.e., low fetal fraction, since little evidence is available per genomic site. Genome-wide noninvasive sequencing of the cfDNA in maternal plasma was shown to reveal the entire fetal genome (Fan et al., 2012). However, for maternal-only heterozygous positions, these methods required maternal haplotype information.

Rabinowitz et al (2019) describe a different approach for genome wide NIPT of monogenic disorders, defining this issue as a unique case of variant calling, termed noninvasive prenatal variant calling. Accordingly, a Bayesian genotyping algorithm utilizes the information of each read, covering each candidate variant, and a machine learning-based fine-tuning step subsequently incorporates information from previously verified results. By accounting for each read, the authors were able to utilize characteristics that separate fetal and maternal DNA, such as fragment length. The algorithm was implemented as Hoobari, the first noninvasive fetal variant caller, that was able to genotype all fetal positions, including biparental loci and indels. However, performance in biparental loci and indels was lower than in positions in which only one parent is heterozygous (WO2021/0340601).

### GENERAL DESCRIPTION

In a first aspect, the present invention provides a method for genotyping a fetus, comprising:
a. receiving reads of sequencing data of (i) maternal cell-free DNA (cfDNA), and (ii) maternal and optionally paternal genomic DNA (gDNA) from a pair parenting the fetus;
b. identifying potential genomic sites at which the fetus may have a variant;
c. for each of the potential genomic sites, determining a probability that the fetus has the variant;
d. generating haplotype phase sets using the maternal gDNA sequencing data, and optionally also paternal gDNA sequencing data; and
e. combining the haplotype phase-sets data with the probabilities obtained in step c to determine the most probable haplotype phase set inherited by the fetus; thereby genotyping said fetus.

In one embodiment, said reads of sequencing data are short reads.

In one embodiment, said reads of sequencing data are long reads.

In one embodiment, said reads of sequencing data comprise short reads and long reads.

In certain embodiments, one or both gDNA sequencing data and the cfDNA sequencing data is obtained by a method selected from a group consisting of whole genome sequencing (WGS), whole exome sequencing (WES), next generation sequencing (NGS), targeted sequencing, panel sequencing, gene sequencing, long-read genome sequencing, paired-end sequencing, single end sequencing, and amplicon sequencing.

In one embodiment, WGS or WES data is obtained by deep sequencing.

In one embodiment, determining said probability is based on at least one Sequence Alignment Map (SAM) parameter.

In one embodiment, determining said probability is based at least on an observed template length.

In one embodiment, said step of determining a probability that the fetus has the variant further comprises calculating a total fetal fraction.

In one embodiment, the method of the invention further comprises constructing a fetal size distribution and a maternal size distribution, wherein said determining the probability that the fetus has the variant comprises binning said fetal size distribution and calculating a fetal fraction for each fragment size bin, and calculating, for at least one size and at least one fragment at said at least one site, a probability that said fragment is fetal, based on a fetal fraction of a respective fragment size bin to which said fragment belongs.

In one embodiment, said determining a probability that the fetus has the variant comprises applying a Bayesian procedure.

In one embodiment, said Bayesian procedure comprises prior probabilities calculated using sequencing data of at least one of said parents.

In one embodiment, the method of the invention further comprises recalibration output of said Bayesian procedure using machine learning.

In one embodiment, said determining the probability is performed using fetal variant calling.

In one embodiment, said determining the probability is performed using the Hoobari algorithm.

In one embodiment, said determining the probability comprises fragmentomics-based probability that the sequence read is of fetal origin.

In one embodiment, the method comprises extracting fragmentomic features for each cfDNA read identified as overlapping a potential genomic site where the fetus may have a variant.

In certain embodiments, said fragmentomic features comprise one or more of read quality mapping, read base qualities, fragment length, short/long read ratio, end motifs, cleavage patterns around methylation sites, read endpoint preferred end, DNA/accessibility/nucleosome, distance to nearest nucleosome, transcription factor binding sites, regional fetal fraction, regional sequence composition, read sequence composition, and number of sequence errors in the read.

In one embodiment, said determining the probability comprises multiplying the variant calling based probability with the fragmentomics-based probability to obtain a calculated joint probability.

In certain embodiments, said step of generating haplotype phase sets is performed using read-backed phasing or using population-based phasing, or a combination thereof.

In one embodiment, the said step of generating haplotype phase sets is performed using read-backed phasing of paired-end reads.

In one embodiment, the said step of generating haplotype phase sets is performed using population-backed phasing comprising population reference panels that match the ethnicity of the parents.

In one embodiment, said method further comprises obtaining long haplotype phase sets by combining the haplotype phase sets with a population haplotype reference panel.

In one embodiment, said long haplotype phase sets are chromosome-length haplotypes.

In one embodiment, said step of combining the haplotype phase-sets data with the obtained probabilities to determine the most probable haplotype phase set inherited by the fetus comprises defining a sliding window around each variant and determining which of the four possible haplotype combinations, from the two maternal and two paternal predicted haplotypes, is present in the read.

In one embodiment the sequencing data of the maternal cell-free DNA (cfDNA), is obtained from a sample comprising a low fetal fraction.

In certain embodiments, said variant is selected from a group consisting of single nucleotide polymorphism (SNP), insertion/deletion (Indel), aneuploidy, copy number variation (CNV), STR (short tandem repeats) variants, expansion mutations, and de novo mutations.

In another aspect, the present invention provides a computer software product, comprising a computer-readable medium in which program instructions are stored, which instructions, when read by a data processor, configure the data processor to (1) receive reads of sequencing data of (i) maternal cell-free DNA (cfDNA), and (ii) maternal and optionally paternal genomic DNA (gDNA) from a pair parenting a fetus, and to (2) execute the method in accordance with the invention.

In another aspect, the present invention provides a system for genotyping a fetus, comprising: an input utility for receiving reads of sequencing data of (i) maternal cell-free DNA (cfDNA), and (ii) maternal and optionally paternal genomic DNA (gDNA) from a pair parenting a fetus; and a data processor configured for analyzing said data for executing the method in accordance with the invention.

### BRIEF DESCRIPTION OF THE DRAWINGS

For better understanding the subject matter that is disclosed herein and to exemplify how it may be carried out in practice, embodiments will now be described, by way of non-limiting example only, with reference to the accompanying drawings, in which:

FIG. 1 is a flowchart diagram of a method suitable for fetal genotyping, according to various exemplary embodiments of the present invention.

FIG. 2 is a schematic representation showing phase set detection. Phase-set 1 is indicated in bold, and phase-set 2 in italics.

FIGS. 3A-3B are graphs showing Negative Predictive Value (NPV) (3A) and Positive Predictive Value (PPV) (3B) for all the tested families following phase-set correction. The values were calculated over 600K-1M maternally inherited SNVs. The intensity of the lines represents the fetal fraction (FF). MV: majority vote, JP: joint probabilities.

FIGS. 4A-4F are graphs showing Positive Predictive Values (PPV) (4A-4C) and Negative Predictive Values (NPV) (4D-4F) for all the tested families, with correction using haplotype majority, and without correction (none). (4A) maternally inherited variants (4B) bi-allelic (Biparental) variants and (4C) Paternally inherited variants. (4D) maternally inherited variants (4E) bi-allelic (Biparental) variants and (4F) Paternally inherited variants. The intensity of the lines represents the fetal fraction (FF).

FIGS. 5A-5F are graphs showing PPV (5A-5C) and NPV (5D-5F) values for maternally inherited (5A and 5D), paternally inherited (5B and 5E) and homozygous biallelic (biparental) variants (5C and 5F) calculated using plasma cfDNA sequencing data down-sampled to different sequencing depths. Results are shown for the baseline hoobari algorithm (light-colored lines) and for haplotypes-based improvements (dark-colored lines).

### DETAILED DESCRIPTION OF EMBODIMENTS

In the NIPT approach which is defined as noninvasive prenatal variant calling as described by Rabinowitz et al (2019), each genetic variant is analyzed independently, and the information that can be deduced from the biological dependance between variants is disregarded.

The present invention is based on the finding that deep whole genome sequencing (WGS) of cfDNA extracted from maternal plasma during pregnancy and its analysis using a variant calling approach and optionally fragmentomics-based analysis, combined with identification of haplotypes, improves the overall accuracy of genotype predictions (e.g., reduces mistaken and low confidence predictions), and enables the identification of various genetic variants in the fetal genome. Surprisingly, fragmentomics information was found to be highly valuable for predicting the most likely haplotype to be inherited by a fetus.

The present invention thus provides a method for genome-wide noninvasive prenatal genotyping that combines both site-level and haplotype-level prediction, optionally utilizing fragmentomics and population information. As shown in the examples below, the method of the invention was tested on 17 families with varying fetal fractions and sequencing depths showing highly accurate results. The method of the invention is highly predictive for genomic sites where the mother or both parents are heterozygous; such sites are generally considered as very challenging. Analysis of cfDNA, either in NIPT or liquid biopsy, requires dealing with low DNA amounts and high noise-levels. Hence, in accordance with the invention combining a fragmentomics-based site level method with the added layer of parental haplotype information, as demonstrated herein, results in accurate cfDNA-based fetal genotyping.

The invention therefore concerns the deduction of the most likely haplotype for each pair or combination of variants based on their tendency to be inherited together. Such tendency may be deduced from comparisons with population reference sets or panels.

In accordance with the invention, the incorporation of information from nearby variants strengthens and affirms the fetal genotype predictions.

Accordingly, in an aspect, the present invention provides a method of genotyping a fetus, comprising:
a. receiving reads of sequencing data of (i) maternal cell-free DNA (cfDNA), and (ii) maternal and optionally paternal genomic DNA (gDNA) from a pair parenting the fetus;
b. identifying potential genomic sites at which the fetus may have a variant;
c. for each of the potential genomic sites, determining a probability that the fetus has the variant;
d. generating haplotype phase sets using the maternal gDNA sequencing data, and optionally also paternal gDNA sequencing data; and
e. combining the haplotype phase-sets data with the probabilities obtained in step c to determine the most probable haplotype phase set inherited by the fetus; thereby genotyping said fetus.

“Blood sample” herein refers to a whole blood sample that has not been fractionated or separated into its component parts as well as to a fractionated blood sample. Whole blood is often combined with an anticoagulant such as EDTA or ACD during the collection process but is generally otherwise unprocessed.

“Blood fractionation” is the process of fractionating whole blood or separating it into its component parts. This is typically done by centrifuging the blood. The resulting components are: (a) a clear solution of blood plasma in the upper phase (which can be separated into its own fractions), (b) a buffy coat, which is a thin layer of leukocytes (white blood cells) mixed with platelets in the middle, and (c) erythrocytes (red blood cells) at the bottom of the centrifuge tube in the hematocrit fraction.

“Blood plasma” or “plasma” is the liquid component of blood (the blood component excluding cells). It makes up about 55% of total blood by volume. It is mostly water (93% by volume), and contains dissolved proteins including albumins, immunoglobulins, and fibrinogen, as well as glucose, clotting factors, electrolytes, hormones, carbon dioxide, and cell free DNA.

Blood plasma can be prepared by centrifuging a tube of whole blood in the presence of an anti-coagulant until the blood cells are separated and pulled down to the bottom of the tube. The blood plasma is then poured or drawn off.

Cell-free DNA (cfDNA) also referred to as “circulating free DNA” are DNA fragments existing outside of cells in vivo circulating in body fluids such as blood plasma. The fragments of cfDNA typically have lengths ranging from about 150 to 200 base pairs (bp), and averaging about 170 bp, which presumably relates to the length of a DNA stretch wrapped around a nucleosome. During pregnancy, cell-free fetal DNA can be found circulating in maternal plasma. Thus, the cfDNA in maternal plasma is a mixture of both maternal and fetal DNA; both the total amount of cfDNA, and the fraction of fetal DNA within it, increases throughout pregnancy.

The term cfDNA also refers to fragments of DNA that have been obtained from the in vivo extracellular sources and separated, isolated, or otherwise manipulated in vitro. cfDNA can be obtained by extracting DNA from blood plasma after removal of intact cells. Methods for extracting cfDNA are well known in the art, for example, as shown in the Examples below.

In addition to the sequencing of cfDNA, paternal and maternal genomic DNA data is also obtained by sequencing DNA derived from a cell-containing sample using a WGS approach, to assign prior probabilities to plasma sequencing reads as to their origins (fetal/maternal).

The term “genomic DNA” or “gDNA” herein refers to DNA existing in a cell in vivo and containing a complete genome of the cell or organism. The term also refers to DNA that has been obtained from the in vivo cell and separated, isolated, or otherwise manipulated in vitro. Typically, the cell is isolated prior to being subjected to lysis to produce in vitro cellular DNA. The term gDNA as used herein does not include cfDNA. The term “sample” herein refers to a sample typically derived from a biological fluid, cell, tissue, organ, or organism comprising a nucleic acid or a mixture of nucleic acids comprising at least one nucleic acid sequence that is to be analyzed for the presence of a genetic variant. Such samples include but are not limited to blood or a blood fraction (for example, peripheral blood mononuclear cells) obtained from whole blood samples. The sample is preferably obtained from a human subject.

The sample may be used directly as obtained from the biological source or following a pretreatment to modify the character of the sample. The sample may be used fresh or thawed after being frozen.

The DNA is extracted using standard protocols, e.g., as described in the examples below.

The term “genomic site” refers to a unique position in the genome. It may be represented by a chromosome ID, chromosome position and orientation on a reference genome.

As used herein the term “haplotype” refers to a set of DNA variations, or polymorphisms, that are located at such physical proximity on the chromosome that they tend not to recombine, and therefore tend to be inherited together.

The sequencing data for the mother and father are used to generate phase-sets for the parents. These phase-sets may be used as such or may be used to deduce longer, for example, but not limited to, chromosome-length parental haplotypes. The deduction of the longer parental haplotype phase sets, e.g., the chromosome-length parental haplotypes, may be based on comparisons with population reference haplotype databases. Alternatively, long-read sequencing can be used to construct the longer, chromosome-length, haplotypes.

As used herein, the “long (or longer) haplotype phase sets” refers to any length of a haplotype that is longer than the originally defined haplotype. In an embodiment, the longer haplotype is a chromosome-length haplotype, also referred to as “full chromosome haplotype”.

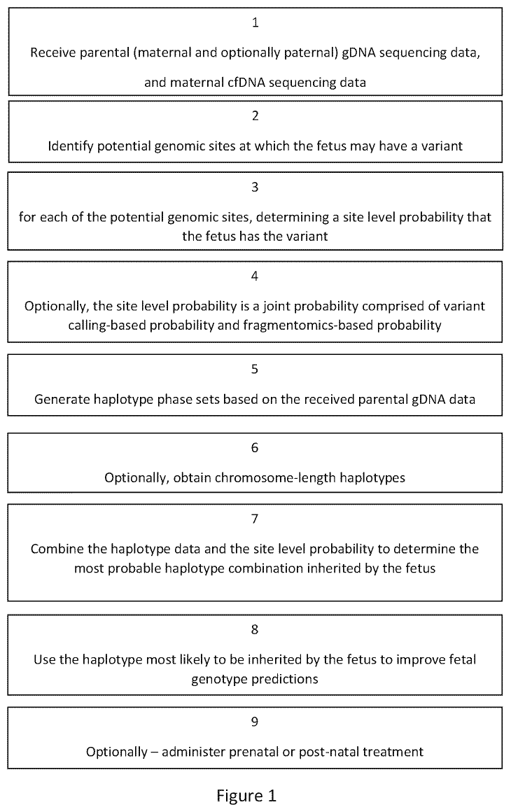

These phase-sets or full-chromosome haplotypes are used, along with probabilities that are assigned to each potential fetal variant (also referred to as “site-level prediction”), for example by an algorithm, e.g., the algorithm Hoobari, as described in (Rabinowitz et al., 2019) and WO2021/0340601, to predict which is the most likely haplotype combination to be inherited by the fetus in every genomic position. Furthermore, this haplotype prediction is used to correct conflicting predictions made by the algorithm Hoobari. A flowchart describing this process is presented in FIG. 1.

The invention thus provides a combination of site-level prediction with haplotype prediction.

The site-level prediction can also be based on analysis of fragmentomic features of the DNA reads.

The maternal genomic DNA (gDNA) data, maternal cell-free DNA (cfDNA) data, and optionally, the paternal gDNA data are obtained by a sequencing method including, but not limited to, deep whole genome sequencing (WGS), whole exome sequencing (WES), next generation sequencing (NGS), targeted sequencing, panel sequencing, gene sequencing, long-read genome sequencing, paired-end sequencing, single end sequencing, and amplicon sequencing.

The term “Next Generation Sequencing” (NGS) herein refers to sequencing methods that allow for massively parallel sequencing of clonally amplified molecules and of single nucleic acid molecules. Non-limiting examples of NGS include sequencing-by-synthesis using reversible dye terminators, and sequencing-by-ligation.

Deep sequencing refers to sequencing a genomic region multiple times, sometimes hundreds or even thousands of times. Deep sequencing of the genome allows researchers to detect rare genetic variants.

As used herein the term “deep whole genome sequencing” refers to deep sequencing of the entire genome. In the context of the present invention cell-free DNA extracted from maternal blood plasma during pregnancy is subjected to deep whole genome sequencing. The maternal blood plasma samples may be obtained at any stage of the pregnancy, preferably between weeks 7-38 of the pregnancy.

The sequencing is repeated multiple times, for example, but not limited to between 10 times (10×) and 1000 times (1000×), e.g., 10 times (10×), 20 times (20×), 30 times (30×), 50 times (50×), 100 times (100×), 150 times (×150), 200 times (200×), 300 times (300×), 500 times (500×), or 1000 times (1000×).

As shown in the Examples below, plasma cfDNA was subjected to varying sequencing depths.

In one non-limiting example, the cfDNA in maternal plasma is sequenced 300 times (300×); in other embodiments, the cfDNA in maternal plasma is sequenced 50 times (50×), 100 times (100×), or 150 times (×150). As described in the Example below, the NPV and PPV nearly plateaued in a sequencing depth as low as 50× for paternal variants, 100× for maternal variants, and 150× for biparental variants. Namely, in accordance with the method of the invention, accurate predictions can be obtained even when lower sequencing depths of the cfDNA are used.

In addition, genomic maternal and, optionally, paternal DNA is also subjected to whole genome sequencing. Such genomic DNA may be obtained from any cell type, for example from blood cells, e.g., leukocytes. In an embodiment, whole genome sequencing of paternal and maternal genomic DNA is performed to a targeted depth of between about 20× and 40×, for example 30×.

Whole genome sequencing may be performed using any method known in the art, for example, the HiSeq X Ten System (Illumina), HiSeq 4000 (Illumina), nanopore WGS sequencing using MinION device (Oxford Nanopore Technologies), and WGS by Ultima Genomics.

The sequencing generates “reads” which are sequences of DNA fragments of varying lengths. Typically, though not necessarily, a read represents a short sequence of contiguous base pairs in the sample. The read may be represented symbolically by the base pair sequence (in A T C G). It may be stored in a memory device and processed as appropriate. A read may be obtained directly from a sequencing apparatus or indirectly from stored sequence information.

The sequencing input may be of long reads (e.g., from about 1 KBP (kilogram base pairs) to about 100 KBP, or more) or short reads (e.g., from between about 50 base pairs and 400 base pairs), or a combination of long reads and short reads.

After sequencing, the reads are aligned to a human reference genome based on sequence similarities.

As used herein, the terms “aligned”, “alignment”, or “aligning” refer to the process of comparing a read to a reference sequence and thereby determining whether the read is contained in the reference sequence. If the reference sequence contains the read, the read may be mapped to a particular location in the reference sequence. In some cases, alignment simply tells whether the read is present or absent in the reference sequence.

Optionally, additional information (also referred to herein as “metadata”) pertaining to one or both the parents is also received. The received metadata optionally and preferably includes at least one, more preferably more than one, of the following features: mutation carrier status of the parents, ethnicity of the parents, body mass index (BMI), and week of pregnancy.

As used herein, the term “variant” or “genetic variant” refers to a change (also referred to as a mutation) in the DNA sequence. The method of the invention is suitable for the detection of various types of genetic variants, including, but not limited to single nucleotide polymorphism (SNP), copy number variation (CNV), including insertion/deletion (Indel), and aneuploidy, STR (short tandem repeats) variants, expansion mutations, and de novo mutations.

The term “single nucleotide polymorphism” or “SNP” herein refers to a genomic variant at a single base position in the DNA.

The term “copy number variation” (CNV) herein refers to any structural genome variant in which the amount of a certain genomic sequence is altered either increased or decreased. As such CNV encompasses deletions, insertions, duplications, multiplications, and translocations. CNV also encompasses chromosomal aneuploidies and partial aneuploidies.

The term “aneuploidy” herein refers to an imbalance of genetic material caused by a loss or gain of a whole chromosome, or part of a chromosome.

The term “short tandem repeat variant” or “STR variant” (also known as microsatellites) herein refers to variations in short tandemly repeated (STR) DNA sequences. These STR involve a repetitive unit of 1-6 base pairs, and form series of repetitions with lengths of up to 100 nucleotides.

The term “expansion mutation” herein refers to an increase in the copy number of a repeated unit, commonly a di- or trinucleotide.

The term “de novo mutation” herein refers to germline mutations that newly occurred within one generation. Namely, a new genetic variation that appeared in a fetus while none of the parents carry the mutation.

The identification of the maternal and paternal variants (i.e., variant sites or mutations) can be performed using a variant calling approach, which is generally based on alignment of the DNA sequencing data and the application of a commercially available variant caller.

Sequence alignment techniques that can be used according to some embodiments of the present invention include, without limitation, Burrows Wheeler Aligner (BWA), ABA, ALE, AMAP, anon, BAli-Phy, Base-By-Base, BHAOS/DIALIGN, Bowtie, Bowtie 2, ClustalW, CodonCode Aligner, Comass, DECIPHER, DIALIGN-TX, DIALIGN-T, DNA Alignment, DNA Baser Sequence Assembler, EDNA, FSA, Geneious, Kalign, MAFFT, MARNA, MAVID, MSA, MSAProbs, MULTALIN, Multi-LAGEN, MUSCLE, Opal, Pecan, Phylo, Praline, PicXAA, POA, Probalign, ProbCons, PROMALS3D, PRRN/PRRP, PSAlign, RevTrans, SAGA, SAM, Se-AI, STAR, STAR-Fusion, StatAlign, Stemloc, T-Coffee, UGENE, VectorFriends, and GLProbs.

Exemplary variant callers suitable for the present embodiments include, without limitation, Genome Analysis Toolkit (GATK) and Freebayes. For example, Freebayes can comprise an alignment based on literal sequences of reads aligned to a particular target, not their precise alignment. GATK can comprise: (i) pre-processing; (ii) variant discovery; and (iii) callset refinement. Pre-processing can comprise starting from raw sequence data, e.g., in FASTQ or uBAM format, and producing analysis-ready BAM files; processing can include alignment to a reference genome as well as data cleanup operations to correct for technical biases and make the data suitable for analysis; variant discovery can comprise starting from analysis-ready BAM files and producing a callset in VCF format; processing can involve identifying sites where one or more individuals display possible genomic variation, and applying filtering methods appropriate to the experimental design; callset refinement can comprise starting and ending with a VCF callset; processing can involve using metadata to assess and improve genotyping accuracy, attach additional information and evaluate the overall quality of the callset.

Also contemplated are variant callers such as, but not limited to, Platypus, VarScan, Bowtie analysis, MuTect and/or SAMtools. For example, Bowtie analysis can comprise implementing the Burrows-Wheeler transform for aligning. MuTect can comprise: (i) pre-processing; (ii) statistical analysis; and (iii) post-processing. Pre-processing can comprise an initial alignment of sequencing reads; statistical analysis can comprise using two Bayesian classifiers, one classifier can detect whether a SNP is non-reference at a given site and, for those sites that are found as non-reference, the other classifier can make sure that the normal does not carry the SNP; post-processing can comprise removal of artifacts of sequencing, short read alignments and hybrid capture. SAMtools can comprise storing, manipulating, and aligning sequencing reads stored as SAM files.

In various exemplary embodiments of the invention the method comprises the determination of the probability, for each variant site, to be of fetal origin, also referred to as site level prediction.

In an embodiment, the determination of the probability of the variant to be of fetal origin comprises constructing a fetal size distribution and a maternal size distribution, binning said fetal size distribution and calculating a fetal fraction for each fragment size bin, and calculating, for at least one size and at least one fragment at said at least one site, a probability that said fragment is fetal, based on a fetal fraction of a respective fragment size bin to which said fragment belongs.

As used herein the term “fetal fraction” or “FF” refers to the portion of fetal cfDNA, within the total amount of cfDNA in the maternal blood. The portion of fetal cfDNA within maternal blood (the fetal fraction) varies throughout the pregnancy, and between individuals, hence this is not regarded as a constant but as a variable. Low levels of fetal cfDNA are referred to as a low fetal fraction.

In an embodiment, said determining the probabilities comprises applying a Bayesian procedure. Optionally, said Bayesian procedure comprises prior probabilities calculated using sequencing data of at least one of said parents.

In an embodiment, this procedure further comprises recalibration of the output of said Bayesian procedure using machine learning.

In a specific embodiment the determination of the probability, for each variant site, to be of fetal origin is performed using variant calling, for example using the Hoobari algorithm as described in Rabinowitz et al., 2019 and WO2021/0340601.

The variant calling site-level prediction can optionally be combined with analysis of fragmentomic features of the DNA reads.

The term “fragmentomic features” refers to molecular characteristics of DNA reads, as well as to genomic, epigenetic and alignment features of the DNA read. Fragmentomic features include, but are not limited to, Read quality mapping, Read base qualities, Fragment length, short/long read ratio, DNA fragment end motifs, Cleavage patterns around methylation sites, Read endpoint preferred end, DNA accessibility/nucleosome positioning inference, Distance to nearest nucleosome, Transcription factor binding sites, Regional fetal fraction, Regional sequence composition, Read sequence composition, and Number of sequence errors in the read.

In certain embodiments, the variant calling probabilities and the fragmentomics-based probabilities are multiplied to calculate joint probabilities, for which a maximum-likelihood approach is applied to predict the genotype at each site.

As used herein the term “generating haplotype phase sets (or “blocks”)” or “haplotype phasing” refers to the process of determining haplotypes, i.e., determining which allelic copies of the variants reside on the same copy of the chromosome. This procedure involves statistical estimation of haplotypes from genotype data.

Phasing can be performed in several ways: read-backed phasing is the process of inferring haplotype information by relying on the existence of two or more alternate allele variants in the same sequencing reads. This allows the phasing of only variants that are heterozygous in the sample. Most phasing software tools require either pedigree information, namely sequencing data from a relative of the parents, or long-read sequencing data. Methods for phasing using short reads are scarce. A read-backed phasing process can be performed using software for phasing genomic variants based on DNA sequencing reads. One such tool is Haplotype Assembly for Future-Generation Sequencing Reads (also known as WhatsHap) (Martin et al., 2016).

Another method of phasing that can be used in accordance with the invention is population-based phasing, or statistical phasing. This method relies on either genotyping large cohorts of individuals or using available haplotype reference databases containing thousands of known haplotypes.

For population-based phasing, tailored reference panels that match the ethnicity/ancestry of the parents can be used, such that better phasing is obtained, and rare variants that are more prevalent in the target population may be detected. Tailored reference panels increase the accuracy of the genotype imputation for constructing chromosome-length haplotypes.

Variant co-occurrence data from dedicated databases may also be used to add phasing information for rare variants. For example, the Genome Aggregation Database (gnomAD) which is comprised of ~21M pairs of rare variants that tend to be inherited together and are therefore part of the same haplotype. The variant co-occurence information can be used to improve the variant genotype prediction by tying together the rare, unphased variants with the chromosome-length haplotypes.

Haplotypes and phase sets may also be determined based on short tandem repeats (STR) based markers that can be found in public reference sets.

In an embodiment, the maternal and paternal whole genome sequencing (WGS) data is phased, using the sequencing reads, in a read-backed phasing approach, using the tool WhatsHap (Martin et al., 2016), with default settings. SNPs and indels may or may not be phased together.

Only reads that contain two or more heterozygous variants are used to identify which variants are linked, namely which variants are physically positioned close to one another and likely inherited together.

As used herein the term “heterozygous” refers to different versions (alleles) of a genomic locus. The term “homozygous” refers to the presence of the same versions (alleles) of the genomic locus.

The term “locus” is used to refer to the specific location of a nucleic acid sequence or variant on a reference chromosome.

The phasing process results in a variant call format (VCF) for each parent that contains phasing information for the variants, namely short haplotypes, named “phase-sets”. These are stretches of heterozygous variants that are phased together, namely, these alleles reside in cis on the same copy of the chromosome. However, at this stage the orientation of the phase sets with respect to other phase sets is unknown.

In one embodiment, the phased sets' orientation with respect to other phase sets is resolved, and other genotypes are imputed using population phasing. Population/statistical phasing tools that can be used according to some embodiments of the present invention include, without limitation, WhatsHap, BEAGLE, SHAPEIT2, SHAPEIT3, SHAPEIT4, Eagle2, HapCup, trioPhaser, and SmartPhase. In one specific example, the identified phase sets are used as input to the tool SHAPEIT4 (Delaneau et al., 2019), which compares the phased sets with a haplotype reference panel in order to deduce chromosome-length haplotypes. Comprehensive reference sets that can be used according to some embodiments of the present invention include, without limitation, Haplotype Reference Consortium (HRC), UK Biobank, 1000 Genomes, and 100,000 Genomes. In one specific example, the reference set is the high coverage 3,202-sample WGS 1 kGP resource, sequenced to a targeted depth of 30×, which also includes 602 complete trios for more accurate phasing (Byrska-Bishop et al., 2021).

Next, the maternal and optionally paternal long haplotype phase sets, for example, the chromosome-length haplotypes, and Hoobari genotype predictions are used to decide, for each variant locus, which is the most probable haplotype combination that was inherited by the fetus. Specifically, a sliding window is defined around each variant and the four possible haplotype combinations, from the two maternal and two paternal predicted haplotypes, are considered.

Multiple computation methods may be used to determine the most probable haplotype phase set (also referred to herein as a haplotype combination) inherited by the fetus, and to correct any mismatched predictions. Following are non-limiting examples of such methods:
(1) A “majority vote” approach—the most probable haplotype phase set is the one matching most of the variant calling, e.g., Hoobari (Rabinowitz et al., 2019) predictions.
(2) Summation of the log of joint variant calling genotype probabilities of Hoobari genotype predictions, as described in (Rabinowitz et al., 2019) and optionally the fragmentomics-based predictions, followed by a maximum likelihood approach to predict the inherited haplotype. The haplotype combination with the largest value is taken. This approach is also referred to as the “joint probability” approach.
(3) A majority vote of machine learning predictions-similar to (1), but instead of using Hoobari raw predictions, predictions corrected by the machine learning model are used (Rabinowitz et al., 2019). As shown in the Examples below, the method of the invention is applicable also for the analysis of maternal samples with a low fetal fraction while using reduced sequencing coverage.

Low FF is a major limitation in any form of NIPT, and this problem intensifies the smaller the tested mutation is, since a smaller number of DNA fragments cover it and can be used to predict its inheritance. Increasing the sequencing depth is often suggested as a solution but holds several limitations of its own: scarce cfDNA levels require PCR amplification or targeted panels, all of which result with errors and bias. Genome-wide 300× coverage was previously suggested, but even though new technologies reduced its cost significantly, it is still costly.

The present invention thus provides a method of genotyping a fetus, wherein the maternal cell-free DNA (cfDNA) is obtained from a plasma sample comprising a low fetal fraction.

The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range.

It must be noted that, as used in this specification and the appended claims, the singular forms “a”, “an” and “the” include plural referents unless the content clearly dictates otherwise.

Throughout this specification and the Examples and claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

Disclosed and described, it is to be understood that this invention is not limited to the specific examples, methods' steps, and compositions disclosed herein as such methods' steps and compositions may vary somewhat. It is also to be understood that the terminology applied herein is used for the purpose of describing specific embodiments only and not intended to be limiting since the scope of the present invention will be limited only by the appended claims and equivalents thereof.

### EXAMPLES

### Materials and Methods

### Sample Collection and DNA Extraction

Samples from each family were collected during week 11-33 of the pregnancy with informed consent. DNA from chorionic villus sampling (CVS) or amniocentesis specimen was extracted using the magLEAD 12 gC, MagDEA Dx kit (ExScale, Chiba, Japan). Peripheral maternal blood was collected using 2-4 Ethylene-diamine-tetra-acetic acid (EDTA) tubes. Within one hour of collection, plasma was separated from blood by centrifugation at room temperature for 10 minutes at 1600×g. The plasma was then centrifuged again at 16,000×g for 10 minutes at room temperature to remove any residual cells. Extraction of cfDNA was performed using the QIAamp Circulating Nucleic Acid Kit (Qiagen). Removal of excess salts resulting from cfDNA purification was conducted using Agencourt AMPure XP beads (Beckman Coulter, Inc.) at a 2× ratio to cfDNA volume. Parental (maternal and paternal) genomic DNA was extracted from peripheral blood mononuclear cells (PBMC) using a standard protocol that includes (i) buffy coat separation and (ii) DNA purification using the magLEAD 12 gC, MagDEA Dx kit (ExScale, Chiba, Japan) according to the manufacturer's instructions.

### Library Preparation and Sequencing

Library preparation was performed using the TruSeq DNA PCR-Free Library Prep Kit (Illumina), for genomic DNA, and the Accel-NGS 2S PCR-free Library Prep Kit for cfDNA samples, according to the manufacturer's instructions. This was followed by sequencing using the NovaSeq platform (Illumina) targeting 150-bp paired-end reads across every DNA sample from each family unit.

Both parental and fetal genomic DNA libraries were sequenced aiming for a conventional depth of 30×.

Cell-free DNA samples were not fragmented during library preparation and were sequenced to a requested coverage of 300×, using the NovaSeq platform (Illumina) with 150-bp paired-end reads.

### Alignment to the Genome

Reads were aligned to the Genome Reference Consortium Human Build 38 (GRCh38/hg38) using Burrows-Wheeler v0.7.834 with default parameters. Duplicate reads, resulting from PCR clonality or optical duplicates, and reads mapping to multiple locations were excluded from downstream analysis.

### Variant Calling

Single-nucleotide substitutions and small insertions and deletions were identified using the Genome Analysis Toolkit (GATK) HaplotypeCaller software v4.2.4.0 applying default parameters and Hoobari. Sequence alignment, removal of duplicate read-alignments, parental variant identification, and non-invasive fetal variant calling were executed as outlined in Rabinowitz et al, 2019 and Rabinowitz et al, 2020. HaplotypeCaller was first run on the aligned sequencing data of both parents together, then on the aligned data of the CVS or amniocentesis samples using the variant sites that were identified in the parental genomes.

Only variants with a read depth ranging from 8 covering reads for the maternal, paternal, and placental samples to between 100 and 1000 covering reads for the plasma sample were considered. Variants were excluded if they exhibited a Hoobari-Phred quality score (Rabinowitz et al, 2019) below 20, did not pass GATK VQSR (Van der Auwera et al, 2013), or displayed Mendelian inconsistencies, where the true fetal genotype from CVS/amniocentesis is impossible given the parental genotypes. Between 80-95% of the variants were preserved for analysis across the various families.

### Pre-Processing of Cell-Free DNA Data

HaplotypeCaller was run on the cfDNA sample only at variant sites that were identified in the parental genomes. Using Hoobari, the allele that was observed by each read, together with the read insert-size, was saved in a separate database.

### Noninvasive Fetal Variant Calling

Hoobari was run using the parental variants and the cfDNA pre-processing results database as input. The output was a standard variant call format (VCF) file. The analysis of the results was held using several software dedicated for VCF manipulation, such as vcflib and vcftools.

### Bayesian Noninvasive Genotyping

At each site of interest, a Bayesian calculation was applied. For each possible fetal genotype:

$$P ( G \vert data ) = \frac{P ( data \vert G ) P ( G )}{{\sum }_{i = 1}^{n} P ( data \vert {G}_{i} ) P ( {G}_{i} )}$$

where G is the fetal genotype and Gᵢ is the ith possible fetal genotype out of n possibilities. For bi-allelic variants, it would be either homozygous for the reference allele (AA), heterozygous (Aa), or homozygous for the alternate allele (aa). P (G) is the prior probability for each genotype and was calculated by Mendelian laws. The data variable denotes the reads that cover a site and P(data|G) denotes the likelihood function, which is defined in this Example as a product of the likelihood of each read:

$$P ( data \vert G ) = {\prod }_{j = 1}^{m} P ( {r}_{j} \vert G , {G}_{M} , f ) = {\prod }_{j = 1}^{m} ( P ( {r}_{j} \vert fet ) P ( fet ) + P ( {r}_{j} \vert mat ) P ( mat ) ) .$$

The likelihood of a read rⱼ depends on the fetal genotype and is calculated using the maternal genotype and the fetal fraction. P(rⱼ|fet) and P(rⱼ|mat) are the probabilities of a read-observation that supports a certain allele, given that the read is fetal or maternal, respectively. This depends on the tested fetal genotype Gᵢ, the maternal genotype G_{M} and the observed allele. P(fet) and P(mat) are the probabilities of observing a fetal or maternal read based only on the fetal fraction, and regardless of the allele that it supports. In order to utilize the size differences between fetal and maternal fragments, the fetal fraction used for each read was calculated only from reads with the same fragment size. For reads that are not properly paired or have a fragment size of >500, the total fetal fraction is used.

### Example 1: Haplotype Phasing of WGS Data

Sequencing data was obtained as described in Materials and Methods above for 17 families.

Short-read next-generation sequencing (NGS) data was used to reconstruct parental haplotypes, i.e., to phase the parental DNA such that information from neighboring variants could be incorporated into the analysis.

Accordingly, the maternal and paternal variants, both SNPs and indels, were phased using WhatsHap v1.4, an open-access software tool for phasing genomic variants using DNA sequencing reads through a read-backed phasing approach (Martin et al., 2016), with default settings. Using this approach, only reads that contained two or more heterozygous variants were used to identify which variants are linked, namely, to identify which alleles are inherited together in a haplotype.

To test the feasibility of short-read based phasing, sequencing data of 17 families was used. For each family maternal and paternal genomic DNA, maternal plasma cfDNA and fetal samples were used. The maternal and paternal samples were phased and statistics concerning the deduced haplotypes were extracted.

This step resulted in the generation of “phase sets” also referred to as “blocks” which are stretches of heterozygous variants that are phased together, like haplotype blocks, showing which of the alleles reside in cis position on the same chromosome.

Overall, for each sample, ~400K phase sets were generated with a median length of 400 bp and a median of 4 phased variants per phase-set. Importantly, even though short sequencing reads were used for the read-backed phasing and not long reads which are optimal for the phasing method, around 80% of the total heterozygous variants in each dataset were included in a phase-set.

### Example 2: Haplotype-Based Corrections of Fetal Genotype Predictions

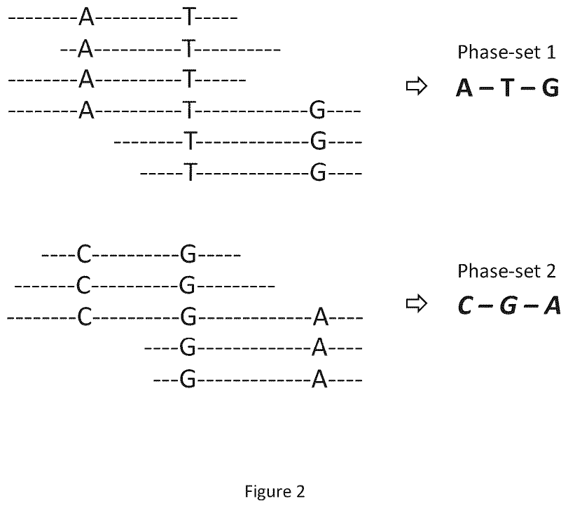

cfDNA was obtained from maternal plasma, and gDNA was obtained from maternal and paternal PBMCs. The DNA was sequenced (by WGS) and subjected to fetal genotype prediction by fetal variant calling using a previously described method (Rabinowitz et al., 2019), and to phase-set detection using the maternal sequencing data as described above (FIG. 2). The phase sets were then used to locate and correct errors in the fetal genotype prediction. Table 1 shows an exemplary correction of an error in fetal phenotype prediction.

TABLE 1

| | | | | Correct genotype | |
|---|---|---|---|---|---|
| Maternal PS | Paternal | Potential | Potential | predictions based | |
| (PS1\|PS2) | positions | fetal PS1 | fetal PS2 | on probable PS | |
| 1\|0 | 0/0 | 1\|0 | 0\|0 | 1/0 | 1/0 |
| 0\|1 | 0/0 | 0\|0 | 1\|0 | 0/0 | 0/0 |
| 1\|0 | 0/0 | 1\|0 | 0\|0 | 1/0 | 1/0 |
| 0\|1 | 0/0 | 0\|0 | 1\|0 | 0/0 | 0/0 |
| 0\|1 | 0/0 | 0\|0 | 1\|0 | 0/0 | 0/0 |
| 1\|0 | 0/0 | 1\|0 | 0\|0 | 0/0 → | 1/0 |
| 0\|1 | 0/0 | 0\|0 | 1\|0 | 0/0 | 0/0 |

The column showing Maternal PS (phase set) shows for each position the presence or absence of a phase set (PS1/PS2). For each maternal phase-set, loci where the father does not carry the alternate allele were considered (see column showing Paternal positions). As shown in the columns Potential fetal PS1 and PS2, for each maternal phase-set, the two potential haplotypes were considered, and the most probable haplotype is chosen based on the fetal variant caller predictions. Discrepancies between the predicted haplotype and the fetal variant caller predictions are corrected, indicated by an arrow in the last column.

Naturally, the shorter the reads are, the chances of finding more than one variant on the same read are lower. For this reason, read-backed phasing works best with long-reads. However, using paired end reads added valuable information since they could be treated as one read.

Next, the phase-sets were used as part of an integrated site-level and haplotype-level method. First, the prior probability of each genotype at each given genomic site was calculated using the parental genotypes (Rabinowitz et al., 2019). The following parameters were then calculated: the likelihood of each read to support each possible fetal genotype, depending on the allele supported by the read, the fragmentomics-based probability that the read is fetal, the maternal genotype at the site, and the tested fetal genotype (Rabinowitz et al., 2019). The prior probabilities and the likelihoods are multiplied by each other to calculate the joint probabilities, for which a maximum-likelihood approach is applied to predict the genotype at each site. In other words, fragmentomics-based read-likelihoods are multiplied to calculate genotype likelihoods; the next step in the integrated method is to multiply genotype likelihoods (or more specifically, joint probabilities) to calculate the haplotype probability. Thus, fragmentomics information uniquely assists in predicting the most likely haplotype.

For each maternal phase set, positions were considered where the paternal genotype is homozygous for the reference allele, and then the maternal phase-set most probable to be inherited by the fetus was assessed in two ways: (1) Summation of the joint probabilities of Hoobari genotype predictions, followed by a maximum likelihood approach to predict the inherited haplotype. (2) A majority vote approach, where the most probable haplotype combination is the one matching the most Hoobari predictions. Like in smoothing, such an approach can reduce noise caused by outlier genotype probabilities along a haplotype; however, over-reducing such noise could result in loss of information. After determining the most probable phase-set inherited by the fetus, any mismatched site-level predictions were corrected.

Negative predictive values (NPVs) were calculated for genotype predictions over all potential variants for three variant categories separately: maternally inherited, paternally inherited and variants inherited from both parents (“homozygous biallelic”). The NPV is the likelihood that a negative prediction concerning the presence of a specific variant in the fetus truly reflects the actual genotype, namely that the fetus does not have the specific genetic variation.

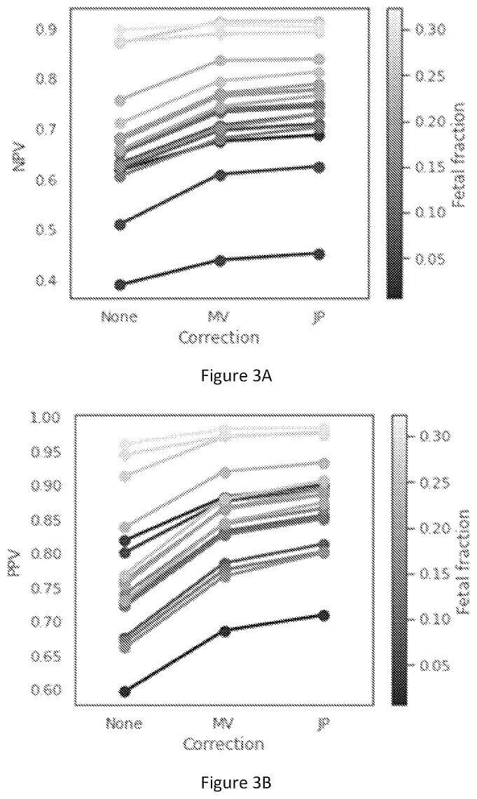

In all the families, both the negative and positive predicted values (NPV and PPV) were prominently improved using both the majority vote approach and the maximum joint probability approach, with a slight advantage for the latter (FIGS. 3A and 3B). Still, differences in overall NPV and PPV can be seen between families with different fetal DNA fraction (FF) values. Of note, the absolute improvement of the results is more prominent for samples with low FF and overall lower baseline performance.

While the read-backed method produced improved results compared with the site-level approach, it is still limited because only a subset of the variants is included in phase-sets. One more caveat is homozygous biallelic variants, i.e., variants that appear in both parents and may result in homozygous mutations. These were not captured easily since both the maternal and the paternal phase-sets at the position are needed to predict the inherited haplotype. However, read-backed phasing using short reads results with maternal phase-sets that do not necessarily overlap the paternal ones, thus reducing the possible space of variants available for correction even further.

Accordingly, the reconstruction of haplotypes that are longer and more complete, i.e., cover larger regions of each chromosome, and consist of more variants was sought. While most heterozygous variants in a sample were included in a phase-set, the orientation of the phase sets with respect to one another was still unknown. Thus, to solve the orientation with respect to the other phase-sets and acquire longer, ideally chromosome-length haplotypes, population haplotype data was introduced as an additional source of information.

To implement population information into the method, the phase-sets found using WhatsHap were used as input to SHAPEIT4, a software tool which combines phased-sets with a population-based haplotype reference panel to deduce chromosome-length haplotypes (Delaneau et al., 2019). Both WhatsHap and SHAPEIT4 are highly robust tools, easy to incorporate with Hoobari, which is a variant caller that returns variant call format (VCF) files. The reference set that was used was the high coverage 3,202-sample WGS 1 kGP resource, sequenced to a targeted depth of 30×, which also includes 602 complete trios for more accurate phasing (Byrska-Bishop et al., 2022). Next the maternal and paternal chromosome-length haplotype and Hoobari genotype predictions were used as described for the phase-sets, to decide for each variant locus which is the most probable haplotype combination that was inherited by the fetus. In contrast with the phase-set based method, artificial 5′ and 3′ limits were set to each haplotype, and not all variants in the chromosome were used. This was done to reduce errors that might arise from recombination events and phasing errors, and to reduce calculation complexity. In practice, a sliding window was defined around each variant and the four possible haplotype combinations from the two maternal and two paternal predicted haplotypes were considered. Subsequently, either a majority vote approach or a maximum joint probability approach was applied to predict the inherited haplotype combination. Chromosome length haplotypes enable to easily apply the method of the invention to all different variant inheritance modes, including homozygous biallelic variants.

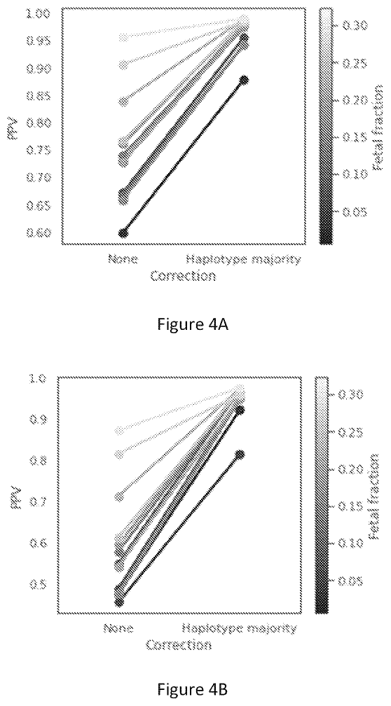

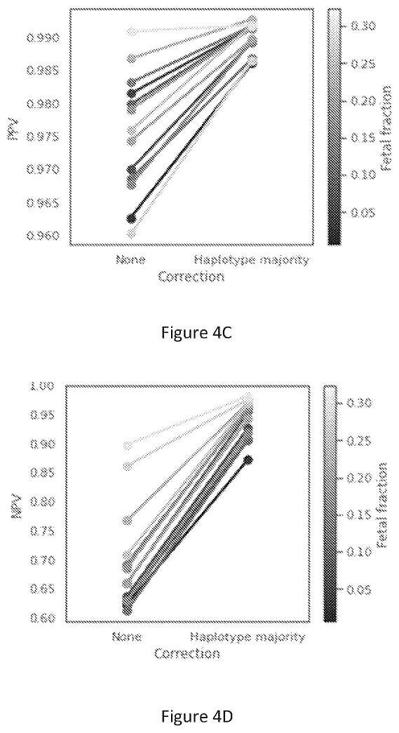

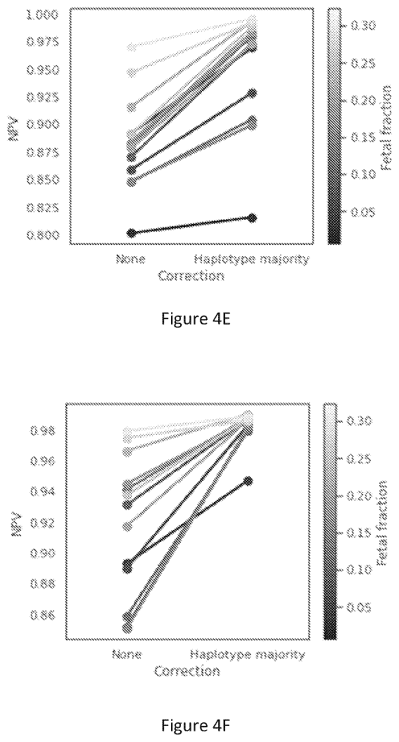

To validate the prediction results, for each family single nucleotide variations (SNVs) were considered; SNVs that can potentially be inherited either from the father, the mother or both parents in each fetus. These SNVs were compared to WGS results of matching samples from pure fetal tissue obtained using amniocentesis or chorionic villus sampling (CVS). The success of the haplotype-based correction approach was assessed by calculating the PPV and the NPV for genotype predictions over all potential variants for the three variant categories separately. For every category, consistent improvement in fetal genotype predictions was observed (FIG. 4).

Haplotype imputation refers to the statistical inference of unobserved genotypes. It is achieved by using known haplotypes in a population, for instance from the HapMap or the 1000 Genomes Project in humans, thereby allowing to test for association between a trait of interest (e.g., a disease) and experimentally untyped genetic variants, but whose genotypes have been statistically inferred (“imputed”).

The following computational approach was used to further improve the prediction accuracy by haplotype imputation. Maternal and paternal phase-sets were imputed using a haplotype reference panel to obtain four separate chromosome-length haplotypes. Using a sliding window approach, all four potential haplotypes were considered, and the most probable haplotype was chosen based on the fetal variant caller predictions. Discrepancies between the predicted haplotype and the fetal variant caller predictions were corrected. Indeed, as shown in FIG. 4, haplotype imputation improved the predictions for all types of variants.

These results demonstrate that adding haplotype information by phasing the maternal and paternal WGS datasets significantly improves fetal genotype predictions.

### Example 3: Predictions Performed on Samples with Low FF

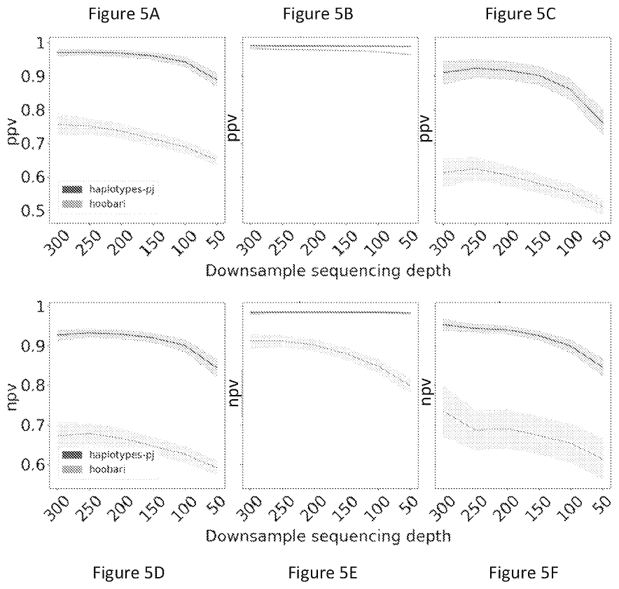

cfDNA samples were down sampled in silico to a range of sequencing depths. The three modes of inheritance (maternal, paternal and biparental) were compared, and the mean NPV and PPV was calculated for the 17 families described above (FIG. 5). On the initial depth of 300×, the integrated haplotype and site level method showed improved NPV and PPV, as already shown. Interestingly, in the integrated method, the NPV and PPV nearly plateaued until a depth of 50× for paternal, 100× for maternal, and 150× for biparental variants. In other words, when haplotype information is available, these values might suffice as the depth of coverage that enables accurate results. Moreover, even at 300×, the site-based approach does not reach the NPV and PPV achieved using the integrated approach at much lower depth values.

1. A method for genotyping a fetus, comprising:
a. receiving reads of sequencing data of (i) maternal cell-free DNA (cfDNA), and (ii) maternal and optionally paternal genomic DNA (gDNA) from a pair parenting the fetus;
b. identifying potential genomic sites at which the fetus may have a variant;
c. for each of the potential genomic sites, determining a probability that the fetus has the variant;
d. generating haplotype phase sets using the maternal gDNA sequencing data, and optionally also paternal gDNA sequencing data; and
e. combining the haplotype phase-sets data with the probabilities obtained in step c to determine the most probable haplotype phase set inherited by the fetus;
thereby genotyping said fetus.

2. The method of claim 1 wherein said reads of sequencing data are short reads, or long reads, or comprise short reads and long reads.

3. (canceled)

4. (canceled)

5. The method of claim 1, wherein one or both of the gDNA sequencing data and the cfDNA sequencing data is obtained by a method selected from a group consisting of whole genome sequencing (WGS), whole exome sequencing (WES), next generation sequencing (NGS), targeted sequencing, panel sequencing, gene sequencing, long-read genome sequencing, paired-end sequencing, single end sequencing, and amplicon sequencing.

6. (canceled)

7. The method of claim 1, wherein determining said probability is based on at least one Sequence Alignment Map (SAM) parameter.

8. The method of claim 1, wherein determining said probability is based at least on an observed template length.

9. The method of claim 1, wherein said step c in claim 1 further comprises calculating a total fetal fraction.

10. The method of claim 9, further comprising constructing a fetal size distribution and a maternal size distribution, wherein said determining the probability of step c in claim 1 comprises binning said fetal size distribution and calculating a fetal fraction for each fragment size bin, and calculating, for at least one size and at least one fragment at said at least one site, a probability that said fragment is fetal, based on a fetal fraction of a respective fragment size bin to which said fragment belongs.

11. The method of claim 1 wherein said determining the probability of step c in claim 1 comprises applying a Bayesian procedure.

12. The method of claim 11 wherein said Bayesian procedure comprises prior probabilities calculated using sequencing data of at least one of said parents.

13. (canceled)

14. The method of claim 1 wherein said determining the probability is performed using fetal variant calling.

15. The method of claim 1 wherein said determining the probability is performed using the Hoobari algorithm.

16. The method of claim 1 wherein said determining the probability comprises fragmentomics-based probability that the sequence read is of fetal origin.

17. The method of claim 16 wherein the method comprises extracting fragmentomic features for each cfDNA read identified as overlapping a potential genomic site where the fetus may have a variant.

18. The method of claim 17, wherein said fragmentomic features comprise one or more of read quality mapping, read base qualities, fragment length, short/long read ratio, end motifs, cleavage patterns around methylation sites, read endpoint preferred end, DNA/accessibility/nucleosome, distance to nearest nucleosome, transcription factor binding sites, regional fetal fraction, regional sequence composition, read sequence composition, and number of sequence errors in the read.

19. The method of claim 16 wherein said determining the probability comprises multiplying the variant calling based probability with the fragmentomics-based probability to obtain a calculated joint probability.

20. The method of claim 1, wherein said step of generating haplotype phase sets is performed using read-backed phasing or using population-based phasing, or a combination thereof.

21. (canceled)

22. (canceled)

23. The method of claim 1, wherein said method comprises obtaining long haplotype phase sets by combining the haplotype phase sets with a population haplotype reference panel.

24. (canceled)

25. The method of claim 1 wherein said step e comprises defining a sliding window around each variant and determining which of the four possible haplotype combinations, from the two maternal and two paternal predicted haplotypes, is present in the read.

26. (canceled)

27. (canceled)

28. A computer software product, comprising a computer-readable medium in which program instructions are stored, which instructions, when read by a data processor, configure the data processor to (1) receive reads of sequencing data of (i) maternal cell-free DNA (cfDNA), and (ii) maternal and optionally paternal genomic DNA (gDNA) from a pair parenting a fetus, and to (2) execute the method according to claim 1.

29. A system for genotyping a fetus, comprising: an input utility for receiving reads of sequencing data of (i) maternal cell-free DNA (cfDNA), and (ii) maternal and optionally paternal genomic DNA (gDNA) from a pair parenting a fetus; and a data processor configured for analyzing said data for executing the method according to claim 1.
